(19) United States
(12) Patent Application Publication
(10) Pub. No.: US 2026/0255073 A1
(43) Pub. Date: Aug. 27, 2026

(54) AN APPARATUS AND METHOD FOR VISUAL INSPECTION

(71) Applicant: Deimos Laboratory Pty Ltd, West Perth, Western Australia (AU)

(72) Inventors: Andrew Donald HADFIELD, Mount Lawley, Western Australia (AU); Peter John HADFIELD, Cottesloe, Western Australia (AU); Jeremy Robert HADFIELD, Mosman Park, Western Australia (AU); Richard MACFARLANE, Cottesloe, Western Australia (AU)

(21) Appl. No.: 18/879,325

(22) PCT Filed: Jun. 30, 2023
(86) PCT No.: PCT/AU2023/050606

§ 371 Date: Dec. 27, 2024

(30) Foreign Application Priority Data
Jun. 30, 2022 (AU) 2022901846

(51) Int. Cl. H04N 23/90; G01N 9/24; G06T 7/00

(52) U.S. Cl. CPC H04N 23/90; G01N 9/24; G06T 7/0002; G06T 2207/20081; G06T 2207/30188

(57) ABSTRACT

An apparatus (10) for visual inspection, the apparatus (10) comprising a receiving means (14) configured to receive a sample of particulates and an imagery means (18) configured to capture imagery of the sample. A method for visual inspection of a sample of particulates is also described.

### FIELD OF THE INVENTION

The present invention relates to an apparatus and method for visual inspection. More particularly, the apparatus and method of the present invention is intended for the visual analysis and quality inspection of grain and other particulates.

Still more particularly, the apparatus and method of the present invention utilises imagery to analyse, inspect and classify grains and other particulates.

### BACKGROUND ART

Standardised inspection of particulates is conducted using imagery to determine defective grains in accordance with Grain Trade Australia (GTA) Trading Standards. For 2021/2022 Trading Standards, Visual Recognition Standards Guide 2021-2022 produced by GTA is used for problem identification.

At present, to visually assess a sample of grain, an individual, for example a sampler or assessor of grain, must be trained to detect defective grains and manually count the defective grains. This practice provides subjective analysis of the sample with different samplers often providing different results, resulting in an inaccurate quality inspection.

In the particulate inspection art, various apparatus are used by samplers during quality inspection of grains and other particulates. Existing apparatus used in the art encompasses two core functions: i) mechanical distribution of the grain and ii) capturing imagery of a single grain kernel for visual analysis after it's been mechanically distributed.

Presently, an apparatus used in the art to inspect quality of grains utilises mechanical distribution of the grain into channels, then feeding the channels under a multispectral line scan sensor and a laser-based depth sensor for visual analysis. Another prior art apparatus inspects grain quality by mechanically distributing the grain into a single line to process one grain kernel at a time, then visually analysing single kernels using an arrangement of mirrors that allows multiple angles of the single kernel to be captured and assessed.

Apparatus presently used in the art generally use mechanical distribution to separate the grains so that imagery of the individual grain kernels is captured. This arrangement is time-consuming, particularly with large samples (e.g. a half hectolitre sample of 16,000 kernels), and reduces the overall productivity of the inspection. However, omission of mechanical distribution is expected to adversely impact the efficiency of standard visual inspections of grain and other particulates.

The Applicant has identified that capturing of individual grain kernels and accordingly, the mechanical distribution to capture the individual grain kernel, may not be necessary in order to perform quality inspections of grains and particulates.

It would be advantageous to provide an apparatus that allowed accurate, consistent and rapid visual analysis for repeatable quality inspections of grains, and further reduced subjective analysis of the sample, reducing the disparate results between different samplers and providing improved accuracy in detecting defective grains.

The apparatus of the present invention has as one object thereof to overcome substantially the abovementioned problems of the prior art, or to at least provide a useful alternative thereto.

Throughout the specification, unless the context requires otherwise, the word “comprise” or variations such as “comprises” or “comprising”, will be understood to imply the inclusion of a stated integer or group of integers but not the exclusion of any other integer or group of integers.

Throughout the specification, unless the context requires otherwise, the word “contain” or variations such as “contains” or “containing”, will be understood to imply the inclusion of a stated integer or group of integers but not the exclusion of any other integer or group of integers.

Each document, reference, patent application or patent cited in this text is expressly incorporated herein in their entirely by reference, which means that it should be read and considered by the reader as part of this text. That the document, reference, patent application, or patent cited in this text is not repeated in this text is merely for reasons of brevity.

Reference to cited material or information contained in the text should not be understood as a concession that the material or information was part of the common general knowledge or was known in Australia or any other country.

### SUMMARY OF THE INVENTION

In accordance with the present invention there is provided an apparatus for visual inspection, the apparatus comprising:
(i) a receiving means configured to receive a sample of particulates; and
(ii) an imagery means configured to capture imagery of the sample.

The particulates comprise of any matter similar in size to a grain kernel, including wheat, barley, oats and canola, as well as rice, nuts and seed. Preferably, the sample of particulates are grains from crops.

Preferably, the receiving means comprises a batch feeder, configured to feed the sample onto a plate.

In one form of the invention, the batch feeder receives a half hectolitre of particulates through a hopper and deposits the sample of the particulates onto the plate.

The plate is preferably formed of glass.

Still preferably, the receiving means further comprises one or more vibration means configured to distribute the sample on the plate. The plate is preferably vibrated in a linear fashion using an exciter.

Preferably, the imagery means comprises at least two capturing elements. The capturing elements are preferably image capturing elements.

Preferably, the image capturing element is adapted to capture a view of the entire sample on the plate, arranged to ensure that no defective grains are missed.

In one form of the present invention, two image capturing elements are arranged in a manner such that the image capturing element captures a top and bottom view of the entire sample on the plate. Preferably, imagery is then captured from each of the two image capturing elements simultaneously. Still preferably, the background for both top and bottom views are uniform. Still yet preferably, the background is black.

In an embodiment, the apparatus further comprises a transfer means configured to move the sample through the apparatus. Preferably, the transfer means comprises a conveyor. Still preferably, the sample on a plate is moved through the apparatus, on a continuous closed-loop conveyor. The conveyor preferably moves the sample through the receiving means and imagery means.

Preferably, the apparatus further comprises a pressurised air cleaning means.

In accordance with a further aspect of the present invention there is provided a method for visual inspection of a sample of particulates, comprising the steps of:
(i) feeding the sample into a receiving means;
(ii) subjecting the received sample into an imagery means to capture imagery of the sample; and
(iii) passing the captured imagery to a processing means to apply one or more data evaluation algorithms on the captured imagery to produce a data output.

Preferably, the data output is information relating to physical features of the sample. Still preferably, the output determines a quality of the sample for visual inspection including presence of defects or contaminants.

In an embodiment of the method, the processing means is adapted to derive the data output using Deep Learning algorithms. The Deep Learning algorithm allows separation of individual grains, even if they are touching each other, within the captured imagery of the complete sample to determine any defects or contaminants in the sample.

### DESCRIPTION OF THE DRAWINGS

The apparatus and method of the present invention will now be described, by way of example only, with reference to two embodiments thereof and the accompanying drawings, in which:—

FIG. 1 is an exploded upper perspective view of an apparatus for visual inspection in accordance with a first embodiment of the present invention;

FIG. 2 is a perspective view of a transfer means of the apparatus of FIG. 1;

FIG. 3 is an exploded perspective view of a batch feeder of the apparatus of FIG. 1;

FIG. 4 is a perspective view of a batch feeder of an apparatus in accordance with a second embodiment of the present invention;

FIG. 5 is an exploded perspective view of a vibrations means of the apparatus of FIG. 1;

FIG. 6 is an exploded perspective view of a vibration means of the apparatus in accordance with the second embodiment of the present invention;

FIG. 7 is an exploded perspective view of an imagery means of the apparatus of FIG. 1 capturing a top view of a sample;

FIG. 8 is an exploded perspective view of an imagery means of the apparatus of FIG. 1 capturing a bottom view of the sample;

FIG. 9 is an exploded perspective view of an imagery means of the apparatus in accordance with the second embodiment of the present invention, capturing a top view of the sample;

FIG. 10 is an exploded perspective view of an imagery means of the apparatus in accordance with the second embodiment of the present invention, capturing a bottom view of the sample;

FIG. 11 is an image captured in the imagery means of the apparatus of FIG. 1 capturing a top view of a sample of wheat;

FIG. 12 is an image captured in the imagery means of the apparatus of FIG. 1 capturing a bottom view of the sample of wheat;

FIG. 13 is an image captured in the imagery means of the apparatus of FIG. 1 capturing a top view of a sample of barley;

FIG. 14 is an image captured in the imagery means of the apparatus of FIG. 1 capturing a bottom view of the sample of barley;

FIG. 15 is an image captured in the imagery means of the apparatus of FIG. 1 capturing a top view of a sample of oats; and

FIG. 16 is an image captured in the imagery means of the apparatus of FIG. 1 capturing a bottom view of the sample of oats.

### BEST MODE(S) FOR CARRYING OUT THE INVENTION

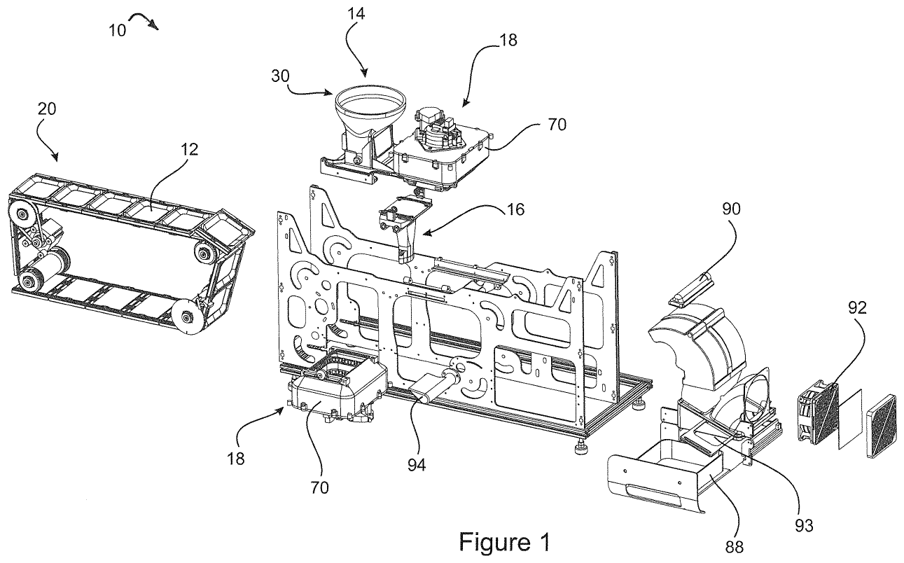

In FIG. 1 there is shown an apparatus for visual analysis and inspection in accordance with a first embodiment of the present invention. The apparatus 10 comprises a receiving means 14 configured to receive a sample of particulates, an imagery means 18 configured to capture imagery of the sample. The sample of particulates being grains which may include wheat, barley, oats and canola.

The receiving means 14 comprises a batch feeder 30 configured to feed the sample onto a glass plate 12. The receiving means 14 further comprises one or more vibration means 16 configured to evenly distribute the sample on the glass plate 12. The glass plate 12 is vibrated in a linear fashion using an exciter 50.

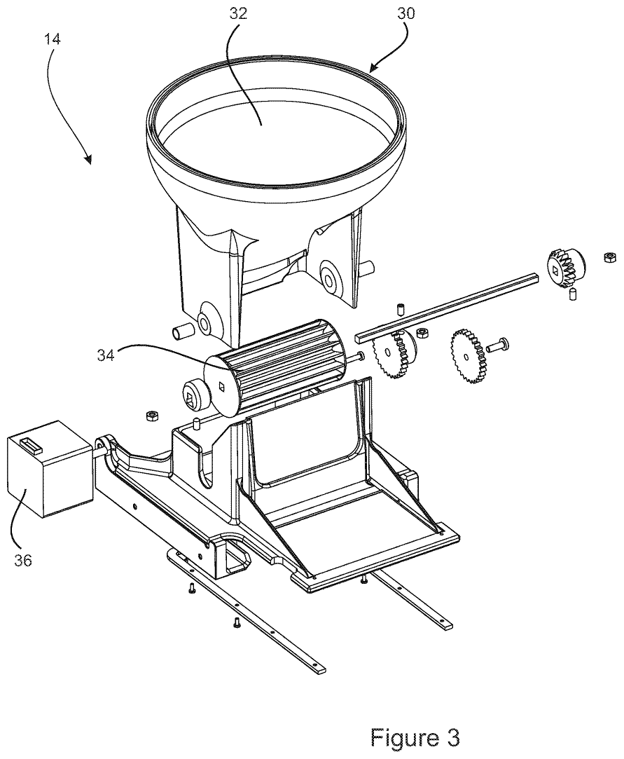

The batch feeder 30 receives an amount for example, half hectolitre of grain into a hopper 32 and deposits a sample therefrom onto the glass plate 12. As shown in FIG. 3, the batch feeder 30 has a hopper 32 which feeds the grain into a feed roller 34 at the bottom of the hopper 32. This feed roller 34 is configured with longitudinal grooves to receive grain therein and to ensure that there are no pinch points that may damage the grains when the feed roller 34 picks up the sample at the bottom of the hopper 32.

The rotation of the feed roller 34 may be adjusted using a motor 36 to optimise the amount of grain that are dispensed onto the glass plate 12.

In accordance with the first embodiment of the present invention, captured imagery from the imagery means, to be described hereafter, is used to determine the density of the sample of the plate. The amount of grain the feed roller 34 captures and rotates may be adjusted in response to the captured imagery, to optimise the amount of grain that are dispensed onto the glass plate 12.

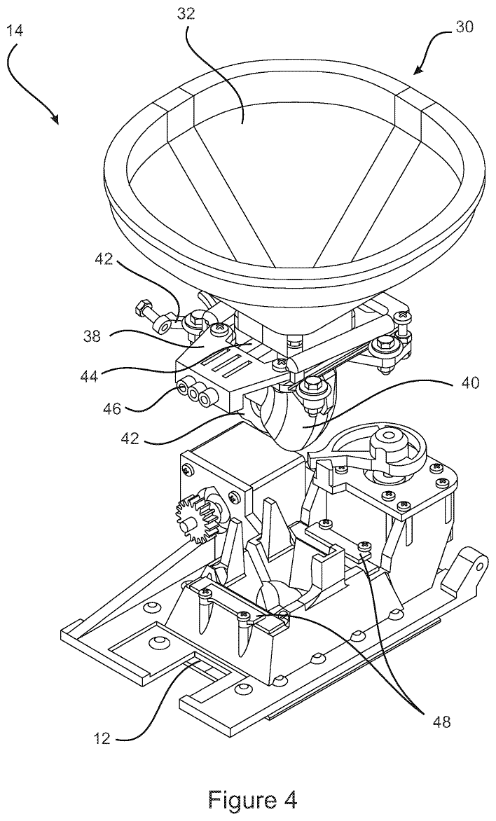

In FIG. 4, there is shown a receiving means 14 in accordance with a second embodiment of the present invention. The batch feeder 30 has a hopper 32 which feeds the grain into a gate 38 at the bottom of the hopper 32. The gate 38 can be opened and closed to deliver grain into a chute 40. The volume of the chute 40 can be adjusted in accordance with the desired volume of the grain sample. The grain in the chute 40 is then released onto the glass plate 12 by dropping a lever plate 42 at the base of the chute 40. The lever plate 42 then returns and the gate 38 opens to accept more grain, and can be repeated as required. Sample size control (or volume control) is provided by the lever plate 42 within the chute 40.

When closing the gate 38 grain may become jammed therein. The gate 38 comprises three gate blades 44 so that when grain does get jammed, only a third of the gate 38 is partially ajar and the potential for grain spilling into the chute 40 is reduced. A spring mechanism 46 is attached to each of the gate blades 44 that provide a tension to remain closed. The spring mechanism 46 ensures that the gate blade 44 is only partially ajar and that the grain is wedged in place. The jammed grain itself provides an obstruction, and thus grain cannot flow through the partially ajar gate blade 44.

The density of the grains on the glass plate 12 impacts the effectiveness of the algorithm used in the processing means (not shown), to be described hereafter.

In accordance with the second embodiment of the present invention, light is used to determine density of the sample on the plate. A light source 48 above the glass plate 12 and a light sensor 63 underneath the glass plate 12 allows the measurement of light penetrating through the glass plate 12 which in turn indicates the density of grains on the glass plate 12. A feedback loop may be required to adjust the number of grains being deposited in order to achieve the optimal density.

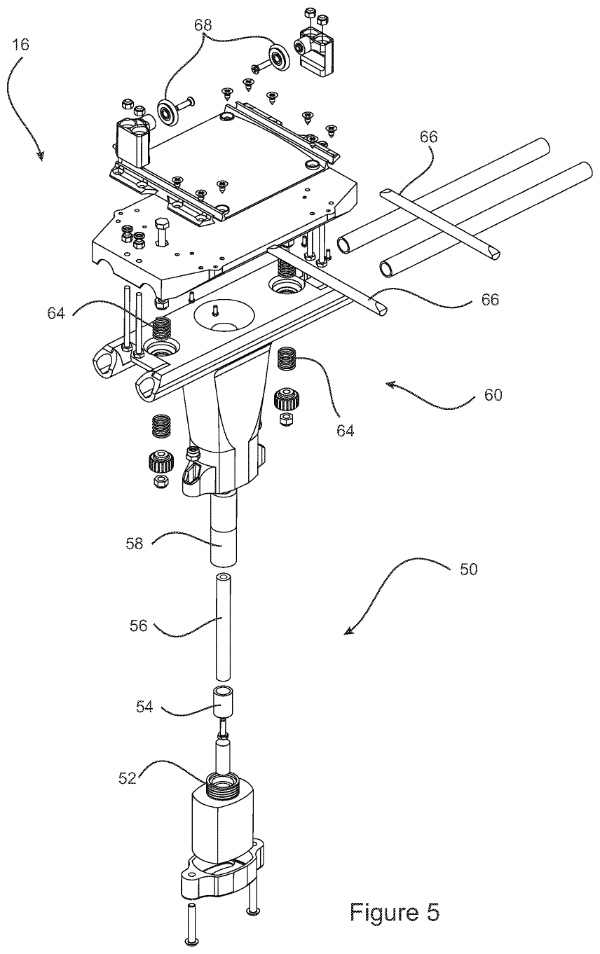

Once the grain has been deposited on the glass plate 12 the grain is then vibrated in a vibration means 16. As shown in FIG. 5, the exciter 50 in accordance with the first embodiment, comprises a solenoid coil 52, a magnet 54, a linear shaft 56 and linear bearings 58, vibrating a vibration cone 60. The solenoid coil 52 may be powered by an alternating current or a waveform via an amplifier, allowing the exciter 50 to reciprocate in a linear fashion, for example up and down, substantially perpendicularly relative to the glass plate 12 (not shown in FIG. 5) in use. An additional benefit of this arrangement is that excessive vibration is not transmitted to the frame of the conveyor 22. The solenoid coil 42 is powered at about 2.5 W, using an alternating current at about 10 V with a frequency of 50 Hz.

In use, the vibration cone 60 vibrates the glass plate 12 using the springs 64 to minimise lateral movement and to ensure that the cone 60 moves in a linear fashion (up and down, or substantially perpendicularly relative to the glass plate 12), and evenly disperse the grains across the glass plate 12. A pair of rods 66, preferably polytetrafluoroethylene (PTFE) rods, sit between the top of the vibration cone 60 and the glass plate 12 to transmit the vibrations from the vibration cone 60 to the glass plate 12. Two groove bearings 68 in the form of rubber wheels are located on the top sides of the glass plate 12 in order to secure the glass plate 12 against the rods 66. When the vibration means 16 is in use the glass plate 12 moves in a linear fashion and the individual grain kernels collide until they are randomly and generally evenly distributed. Additional vibration means may be incorporated in the receiving means 14.

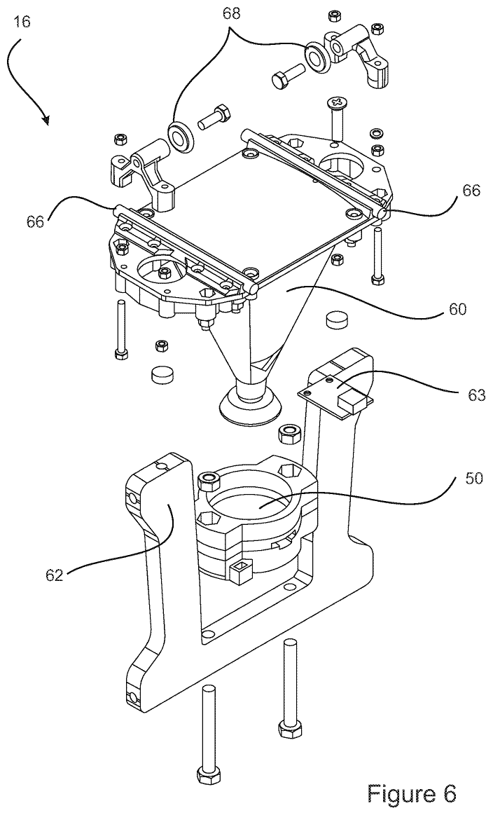

As can be seen in FIG. 6, an exciter 50 in accordance with the second embodiment of the present invention is shown. The exciter 50 vibrates a vibration cone 60. The vibration cone 60 is attached to a frame 62 by way of a spring (not shown) to minimise lateral movement and to ensure the cone 60 moves in a linear fashion (up and down relative to the glass plate 12), with the additional benefit that excessive vibration is not transmitted to the frame of the conveyor 22.

The imagery means 18 comprises at least two image capturing elements 70, adapted to capture a view of the entire sample on the glass plate 12. The at least two image capturing elements 70 are provided to minimise the opportunity for any grain defect to be overlooked, particularly defects that may present only on one side of the grain. By capturing imagery of both sides of the grain, the processing means (not shown), using Deep Learning algorithms for segmentation and classification, separates the individual grains within the captured imagery and performs a comparison of the at least two captured images. As such, highly accurate visual detection of defective grains is achieved.

As shown in FIG. 1, the apparatus 10 comprises of two image capturing elements 70, arranged in a manner such that the two opposing image capturing elements 70 are placed above and below the glass plate 12, capturing a top and bottom view of the sample.

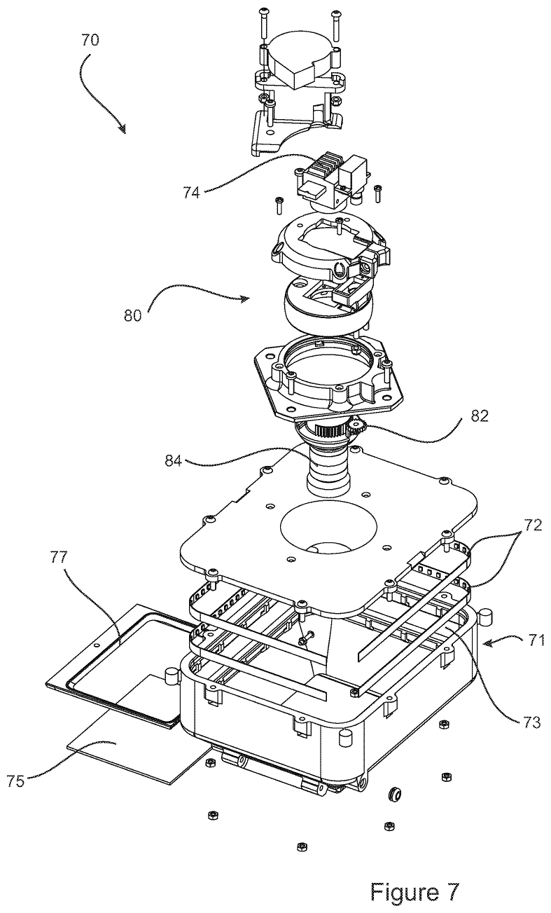

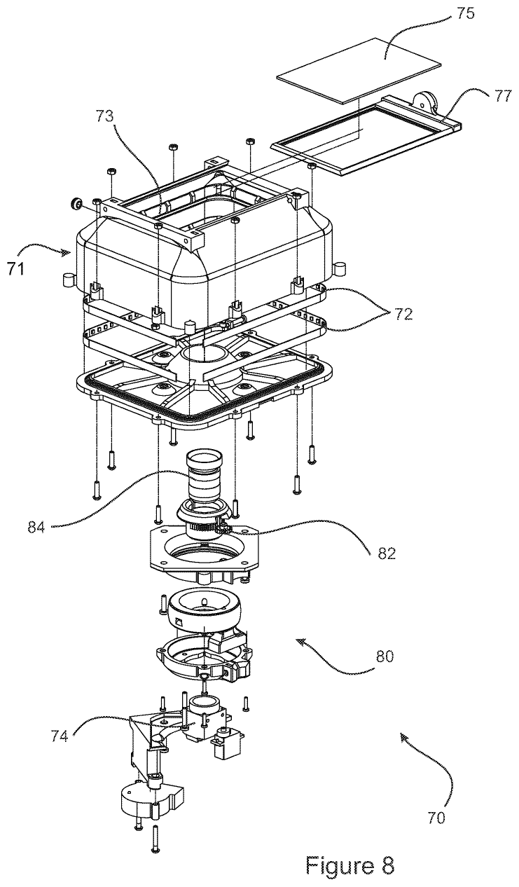

In FIGS. 7 and 8, each of the two top and bottom image capturing elements 70 are shown, respectively. The image capturing elements 70 comprise enclosed boxes 71 further comprising a camera 74, a camera mount 80, lens 84 and a focusing gear mechanism 82. An inside wall 73 of the box is wrapped with two rows of LED lights 72. A camera mount 80, for example in the form of a ball and socket arrangement is provided for precise alignment of the cameras 74 and incorporates one or more servo motors for focus control, including remote and automatic focus controls. A focusing gear mechanism 82 is integrated into the camera mount 80, attached to the lens 84 of each image capturing element 70.

The enclosed boxes 71 are fully sealed and allow a glass plate 75, held in a slidable tray 77 and provided at the bottom/top of the top/bottom image capturing elements 70 to be easily removed for cleaning.

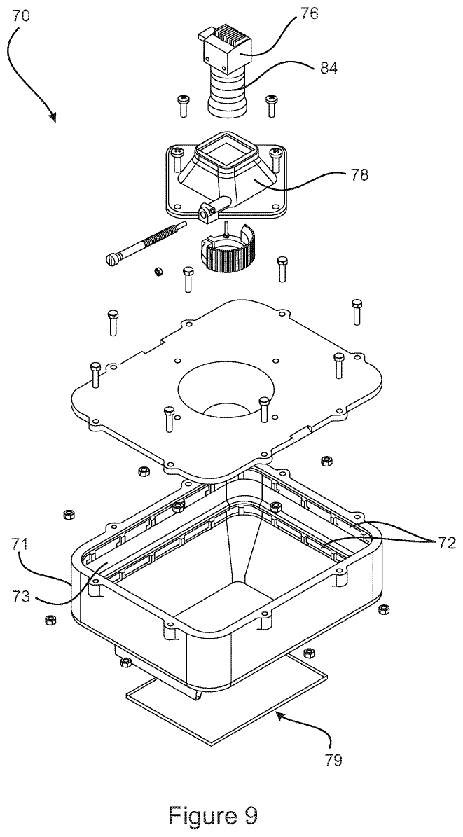

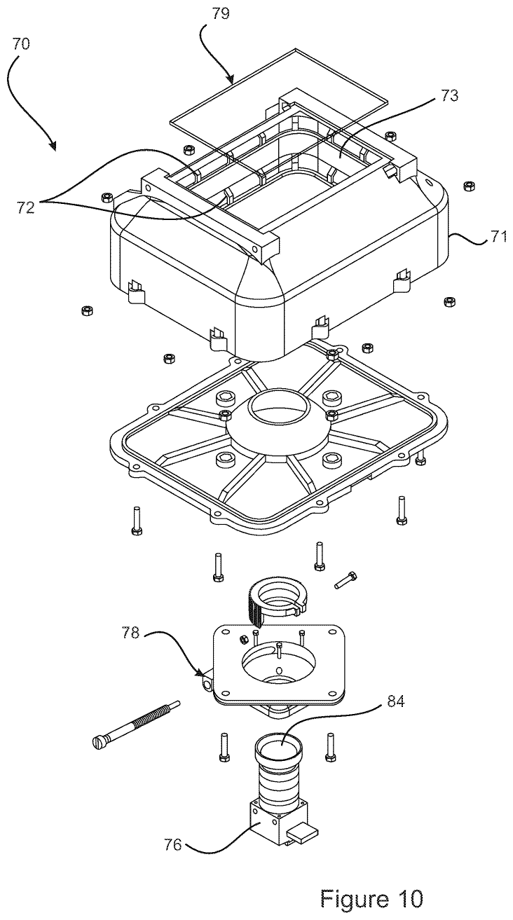

In FIGS. 9 and 10 there is shown the top and bottom image capturing elements 70 in accordance with the second embodiment of the present invention, respectively. The image capturing elements 70 are enclosed boxes 71 with a side 79 of the box closest to the glass plate 12 being transparent and formed of non-reflective glass. The remaining inside walls 73 of the image capturing elements 70 are white and wrapped with two rows of LED lights 72. The LED lights 72 are positioned to avoid light reflected off the glass plate 12 being captured by the image capturing sensor 76. To ensure a uniform background to aid with segmentation, the lens 84 of each image capturing element 70, its mount 78 on the image capturing element 70 (which is for example covered with a matte fabric).

It is important that the image capturing elements 70 are aligned to allow the top and bottom views of the grain kernels to correspond accurately. Imagery is then captured from each image capturing elements 70 simultaneously. As the glass plate 12 is transparent, the background for the top view is the top view of the bottom image capturing element 70 and vice versa for the bottom view of the top image capturing element 70.

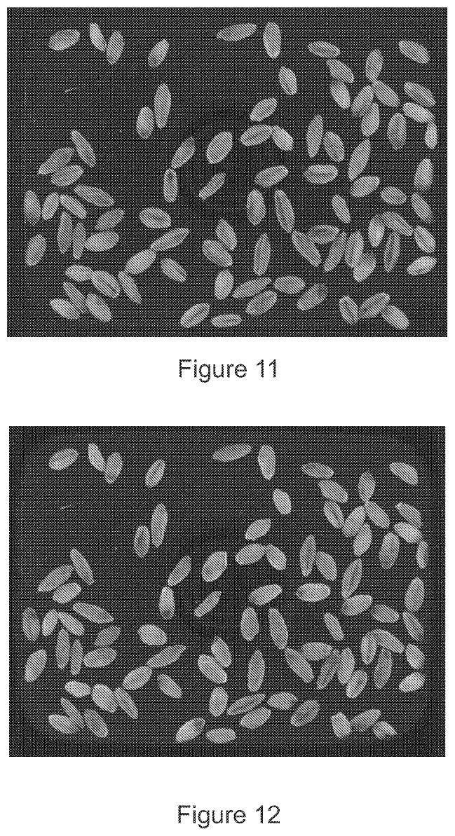

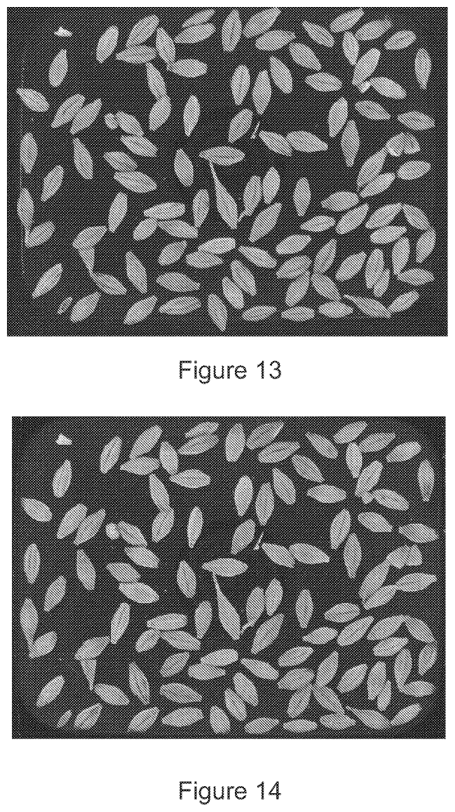

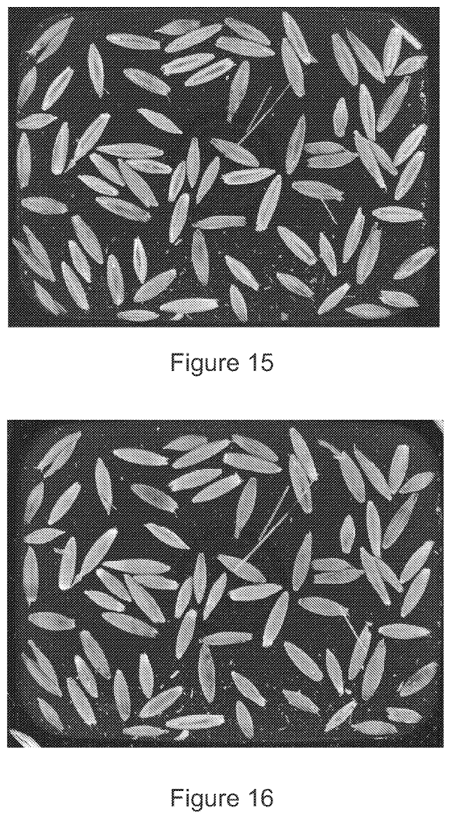

In FIGS. 11 to 16, there are shown images of top and bottom views of each sample of wheat, barley and oats, captured using the two top and bottom image capturing elements 70 of the apparatus 10. One of the views, which may be either top or bottom, has been digitally mirrored.

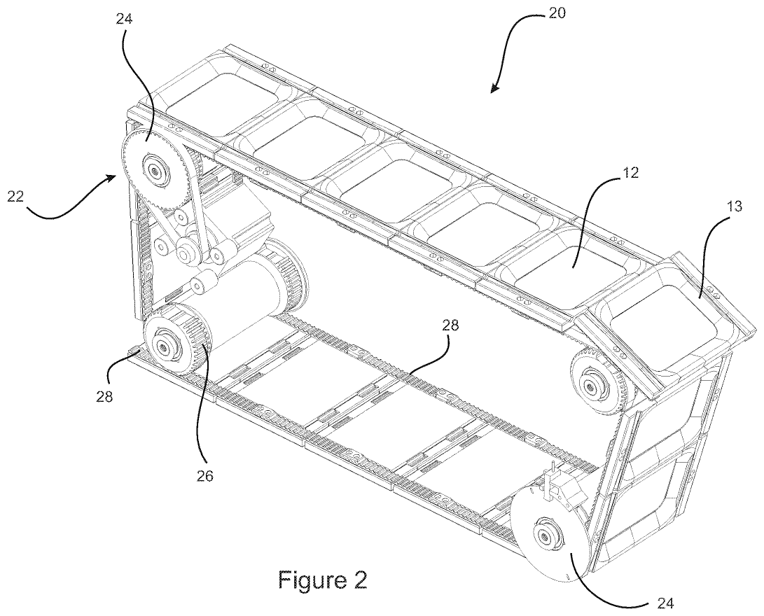

The apparatus 10 further comprises a transfer means 20 configured to move the sample through the apparatus 10. As seen in FIGS. 1 and 2, the transfer means 20 comprises a conveyor 22 which a plurality of glass plates 12 are moved through the apparatus 10 stopping at each means, for example receiving means 14 and Imagery means 18, on a continuous closed loop. The conveyor 22 comprises four rollers 24 each provided with a pair of toothed gears 26 positioned at end thereof.

The conveyor 22 is arranged with fifteen (15) glass plates 12 in a continuous closed loop, each glass plate 12 is positioned in a frame 13, the frame 13 having provided therein a pair of toothed belt sections 28, as shown in FIG. 2. The toothed belt sections 28 are configured to engage with the toothed gears 26.

The conveyor 22 is arranged relative to the receiving 14 and imagery means 18 such that each individual plate 12 and frame 13 pass thereby in sequence.

The apparatus 10 may further comprise one or more pressurised air cleaning means, for example a high pressure blower 90, wherein a row of air jets direct pressurised air onto the glass plate 12. The high pressure blower 90 is positioned near an exit pot 88 so that any remaining debris left on the glass plate 12 is discharged into the exit pot 88. The pressurised air cleaning means may also take form of a blower 94 positioned next to the bottom image capturing element 70, directing air for cleaning the top of the bottom image capturing element 70.

The apparatus 10 may further comprise duct means (not shown) so that air in the apparatus 10 moves towards the exit pot 88. Exhaust fan 92, drawing air out of the apparatus 10, is positioned on top of the exit pot 88. A layer of mesh 93 is positioned between the exit pot 88 and the exhaust fan 92, above the lip of the exit pot 88. The exhaust fan 92 is activated only while the apparatus is in use and directs any light material and dust from the grain towards the exit pot 88. When the apparatus is turned off, the exhaust fan 92 is turned off and any light material that is being drawn against the mesh layer 93 drops back into the exit pot 88.

There is also provided a method for visual inspection of a sample of particulates. The method comprises feeding the sample into a receiving means 12, subjecting the received sample into an imagery means 18 to capture imagery of the sample, and passing the captured imagery to a processing means (not shown) to apply one or more data evaluation algorithms on the captured imagery to produce a data output.

The data output determines if the grain presents any visual defects or whether there are any contaminants in the sample, such as weeds, sands, bugs or other grain types.

The processing means (not shown) is adapted to derive the data output using Deep Learning algorithms. Said Deep Learning algorithm is used for segmentation and classification, presenting data output which determines if the grain presents any visual defects or whether there are any contaminants in the sample. In detail, the processing means (not shown) separate individual grains, even if they are touching each other, within the captured imagery of the complete sample on the glass plate 12.

It is envisaged that the Deep Learning algorithms are provided in the form of a combination of algorithms in conjunction with a significant amount of training (imagery) data.

It is envisaged that the geometry of the hopper 30 and the feed roller 34 of the first embodiment are such that additional sealing mechanisms are not required to retain the grain in the hopper 30.

In an embodiment of the present invention, the exciter 50 in the vibration means 16 is a round audio exciter. Alternative exciters may include devices that use a crank mechanism to provide linear vibration.

In a further embodiment of the present invention, the imagery means 18 may further comprise additional capturing elements. These elements may capture different wavelengths of light to compliment the visible wavelengths (red, green, blue) captured by the image capturing elements.

It is envisaged that the apparatus of the present invention will provide efficient separation of the grains and achieve a high throughput, thus providing an accurate, consistent and rapid visual analysis for repeatable quality inspection of grains.

It is further envisaged that, to maintain accuracy and repeatability, a calibration process may be used by incorporating a calibration plate. For example, the calibration plate will be placed as one of the fifteen (15) plates as shown as an embodiment in FIG. 2 and will be placed with various different objects and/or patterns to ensure that the imagery being captured in the imagery means (both top and bottom capturing elements) will be within tolerance.

It is to be understood that particulates are not limited to grains but may be any matter similar in size to a grain kernel including rice, nuts and seeds.

As can be seen from the above description, the apparatus and method of the present invention provides an accurate, consistent and rapid visual analysis for repeatable quality inspections of grains. Furthermore, the apparatus and method of the present invention reduces subjective analysis of the sample, thus reducing disparate results between different samplers and providing improved accuracy in detecting defective grains.

Modifications and variations such as would be apparent to the skilled addressee are considered to fall within the scope of the present invention.

1. An apparatus for visual inspection, the apparatus comprising:
(i) a receiving means configured to receive a sample of particulates; and
(ii) an imagery means configured to capture imagery of the sample.

2. The apparatus of claim 1, wherein the receiving means comprises a batch feeder, configured to feed the sample onto a plate.

3. The apparatus of claim 2, wherein the batch feeder receives a half hectolitre of particulates through a hopper and deposits the sample of the particulates onto the plate.

4. The apparatus of claim 2, wherein the plate is formed of glass.

5. The apparatus of claim 1, wherein the receiving means further comprises one or more vibration means.

6. The apparatus of claim 5, wherein the vibration means is configured to distribute the sample on the plate.

7. The apparatus of claim 5, wherein the plate is vibrated in a linear fashion using an exciter.

8. The apparatus of claim 1, wherein the imagery means comprises at least two capturing elements.

9. The apparatus of claim 8, wherein the capturing elements are image capturing elements.

10. The apparatus of claim 9, wherein the two image capturing elements are arranged in a manner such that the image capturing elements captures a top and bottom view of the entire sample of the plate.

11. The apparatus of claim 10, wherein the imagery is then captured from each of the two image capturing elements simultaneously.

12. The apparatus of claim 10, wherein the background for both top and bottom views are uniform.

13. The apparatus of claim 12, wherein the background is black.

14. The apparatus of claim 1, further comprising a transfer means configured to move the sample through the apparatus.

15. The apparatus of claim 14, wherein the transfer means comprises a conveyor.

16. The apparatus of claim 14, wherein the sample on a plate is moved through the apparatus, on a continuous closed-loop conveyor.

17. The apparatus of claim 15, wherein the conveyor moves the sample through the receiving means and the imagery means.

18. The apparatus of claim 1, further comprising one or more pressurised air cleaning means.

19. A method for visual inspection of a sample of particulates, comprising the steps of:
(i) feeding the sample into a receiving means;
(ii) subjecting the received sample into an imagery means to capture imagery of the sample; and
(iii) passing the captured imagery to a processing means to apply one or more data evaluation algorithms on the captured imagery to produce a data output.

20. The method of claim 19, wherein the data output is information relating to physical features of the sample.

21. The method of claim 19, wherein the output determines a quality of the sample for visual inspection including presence of defects or contaminants.

22. The method of claim 19, wherein the processing means is adapted to derive the data output using Deep Learning algorithms.

23. The method of claim 22, wherein the Deep Learning algorithm allows separation of individual grains within the captured imagery of the sample.

24. A method for visual inspection of a sample of particulates, the method comprising the steps of:
(i) feeding the sample into an apparatus according to claim 1, to capture imagery of the sample; and
(ii) passing the captured imagery to a processing means to apply one or more data evaluation algorithms on the captured imagery to produce a data output.
